(19) United States
(12) Patent Application Publication
(10) Pub. No.: US 2024/0199565 A1
(43) Pub. Date: Jun. 20, 2024

(54) PROCESS FOR PRODUCING FURFURAL AND CATALYST FOR USE IN SAME

(71) Applicants: MONASH UNIVERSITY, Victoria (AU); ADVANCED FUEL INNOVATION PTY LTD, Sydney (AU)

(72) Inventors: Qiaoqiao Zhou, Victoria (AU); Lian Zhang, Victoria (AU)

(21) Appl. No.: 18/553,529

(22) PCT Filed: Apr. 1, 2022
(86) PCT No.: PCT/AU2022/050296

§ 371 Date: Sep. 29, 2023

(30) Foreign Application Priority Data
Apr. 1, 2021 (AU) 2021900972

(51) Int. Cl. C07D 307/48; B01J 27/053

(52) U.S. Cl. CPC C07D 307/48; B01J 27/053; B01J 2531/26

(57) ABSTRACT

Provided herein is a process for producing furfural from lignocellulosic biomass or an extract thereof, such as an extract rich in saccharides, using solid zinc sulfate-rich catalysts to catalyse conversion to furfural. Also provided herein are zinc sulfate-rich catalysts, and processes for producing zinc sulfate and zinc sulfate-rich catalysts from tyre char.

### FIELD

The present disclosure relates to a process for producing furfural from lignocellulosic biomass or an extract thereof using a solid zinc sulfate-rich catalyst. The present disclosure also relates to zinc sulfate-rich catalysts which find use in such processes, and further relates to processes for producing zinc sulfate and zinc sulfate-rich catalysts from tyre char.

### BACKGROUND

The production of value-added, renewable chemicals from lignocellulosic biomass is crucial for a smooth transition of the current economy into a carbon-neutral future. As a useful precursor and platform material for the synthesis of a broad range of end-products including pharmaceuticals, herbicides, stabilizers, and polymers, furfural has been listed as one of top 12 biomass-derived products recognized by United States Department of Energy (DOE) [1, 2] Traditionally, furfural is produced from C5 hemicellulose by hydrolysis. Compared to the production of furfural from C5 hydrocarbons, the transformation of C6 sugars into furfural is still not successful, due to an extremely low furfural yield for C6 caused by the difference of its composition and structure to C5 [1, 3].

Solvent-thermal reaction, namely solvolysis or hydrolysis, has to date proved to be one of the most efficient approaches to improve the selectivity of chemicals from biomass [1, 4]. In industry, furfural is currently produced from hemicellulose by using concentrated or diluted mineral acid such as H₂SO₄, HCl and H₃PO₄ as catalysts, achieving a furfural yield of 50%-75% at a typical temperature range of 160-240° C. in water [1, 5, 6].

One such example is described in US2011/0144359 [7], which provided a method for producing furfural from lignocellulosic biomass material in a plug flow reactor. In the best case scenario, 5 wt % xylose in water was converted to 67.6 mol % furfural with 0.5 wt % H₂SO₄ as catalyst at 230° C., and in 5.87 min. The overall conversion of xylose was 98.78%. As a further example, US2013/0109869 [8] introduced a metal halide in the water-miscible solutions for a similar furfural production process from lignocellulosic biomass. Under the optimum conditions including the use of 25% corncob, 0.15 M H₂SO₄ and 1% NaCl in a mixed solvent of water (7.3 g), tetrahydrofuran (THF, 42.0 g) and sodium chloride (0.7 g), 160° C. and 30 min, the yield of furfural was reported to reach 87%.

More recently, US2014/0171664 [9] stated a process for producing furfural from a xylan-containing lignocellulosic feedstock. A mixture of corn cob (16 g) and aqueous sulfuric acid (6.4 g) at varying pH was loaded into an auto-clave and subjected to a series of six temperature/pressure cycles. In the best case scenario, the use of aqueous sulfuric acid at pH of 0.50 generated a furfural yield of 68% and a complete conversion of xylan. Another study reported that the highest yield of furfural from glucose was up to 0.69 mol/mole glucose at 180° C. for 33 min, upon the use of modified Sn-Beta zeolites as catalysts in gamma-valerolactone (GVL) solvent [1].

Regardless of its maturity as a process, solvolysis has a number of drawbacks to overcome, including a difficult and costly separation and recovery of furfural, corrosiveness of mineral acid and the large quantity of wastewater derived from the use of mineral acids [10].

There is still a need to reduce the large volume of acid currently used during solvolysis processes due to the potential environmental and handling issues for furfural production.

Across the world, an estimated 1.5 billion vehicle tyres need to be replaced on vehicles every year [11]. Whilst some can be retreaded, a significant proportion of tyres are sent to landfill, and waste vehicle tyres constitute a significant environmental issue. For example, hazardous tyre components can leach into the surrounding environment, and tyre stockpiles present a fire risk.

Vehicle tyres typically contain a complex mix of materials, including natural and synthetic rubber compounds, reinforcing compounds such as steel wire and polymeric fibres, plasticisers, fillers including carbon black and silica, and chemicals for vulcanisation such as sulphur and zinc oxide compounds. This complex mixture adds to the difficulty of recovering and reusing tyre components.

One approach that has been used to produce useful products from waste tyres is pyrolysis of waste tyres [12]. Typically, this involves heating the tyres at high temperatures in a reactor to produce components such as tyre oil, char and gas products. However, there remains a need to identify further uses for waste tyre products, and for processes to recover useful components from scrap tyres.

It would be desirable to provide further processes for the production of furfural which address at least one of the aforementioned issues. It would also be desirable to provide a process for the production of furfural which allow for high conversion of biomass components and/or high selectivity for furfural.

It would further be desirable to find processes which permit efficient recovery of valuable materials from scrap materials such as tyre char.

### SUMMARY OF THE INVENTION

The present inventors have identified that solid zinc sulfate-rich catalysts have strong catalytic effects on the production of furfural from a variety of different biomass components by flash pyrolysis. Both purified zinc sulfate, and transition metal-doped zinc sulfate catalysts have been found to be effective in the process. The present inventors have also found a new process for obtaining zinc sulfate and transition metal-doped zinc sulfate, for example to provide catalysts useful in the production of furfural, from scrap tyre char.

Accordingly, in a first aspect, there is provided a process for producing furfural comprising the steps of pyrolyzing a lignocellulosic material or a fraction thereof and producing furfural, wherein the production of furfural is catalysed by a solid zinc sulfate-rich catalyst.

In some embodiments, the catalyst comprises at least 50 wt % zinc sulfate.

In some embodiments, the solid zinc sulfate-rich catalyst contains a further transition metal. In some embodiments, further transition metal is palladium, iron, copper, cobalt or nickel.

In some embodiments, the further transition metal is palladium.

In some embodiments, the solid zinc-sulfate rich catalyst contains palladium metal and/or palladium oxide.

In further embodiments, the solid zinc sulfate-rich catalyst contains from 0.2 to 5 wt % further transition metal.

In some embodiments, the solid zinc sulfate-rich catalyst contains from 1 to 5 wt % further transition metal.

In some embodiments, the catalyst consists essentially of zinc sulfate.

In some embodiments, the lignocellulosic material or fraction thereof is selected from the group consisting of wood chips, sawdust, sugar cane, corncobs, bagasse, oat hulls, cottonseed hulls, rice hulls and wheat bran.

In further embodiments, the lignocellulosic material or fraction thereof is selected from the group consisting of a cellulosic fraction and a hemicellulosic fraction.

In further embodiments, the lignocellulosic material or fraction thereof is selected from the group consisting of a monosaccharide, a disaccharide and an oligosaccharide.

In some embodiments, the lignocellulosic material or fraction thereof is selected from the group consisting of allose, glucose and xylan.

In some embodiments, prior to pyrolysis, the lignocellulosic material or fraction thereof is subjected to one or more pre-processing steps to either remove a lignin fraction and/or to hydrolyse saccharide linkages.

In some embodiments, the production of furfural is carried out in the absence of steam.

In some embodiments, the production of furfural is carried out in the presence of steam.

In a preferred embodiment, the weight ratio of steam to the lignocellulosic material or fraction thereof is in the range of from about 0.1:1 to about 20:1, optionally from about 8:1 to about 20:1.

In some embodiments, pyrolysis and/or production of furfural is carried out at a temperature in the range of from about 300 to about 500° C.

In some embodiments, the process according to the invention is carried out as a batch process.

In some embodiments, the process according to the invention is carried out as a continuous or semi-continuous process.

In some embodiments, the process according to the invention comprises separating furfural from other reaction products.

In further embodiments, furfural is separated by distillation.

In further embodiments, furfural is separated by selective condensation of a product vapour stream containing furfural.

In another aspect, there is also provided furfural produced by the process as defined herein.

In another aspect, there is provided a process for producing one or more of furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin, comprising producing furfural in accordance with processes according to the invention; and converting the furfural into furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin.

In another aspect, there is provided a process for producing solid zinc sulfate comprising the steps of contacting tyre char with aqueous sulfuric acid and producing a mixture of aqueous zinc sulfate and solid tyre char residue; separating aqueous zinc sulfate from solid tyre char residue; and recovering solid zinc sulfate from the aqueous zinc sulfate.

In a preferred embodiment, the step of contacting tyre char with aqueous sulfuric acid is carried out at a temperature in the range of from about 25 to about 90° C.

In some embodiments, the aqueous sulfuric acid has a sulfuric acid concentration in the range of from about 1 to about 5 moles per litre and used at a mass ratio in the range of from 1.5:1 to 10:1 aqueous sulfuric acid to tyre char.

In some embodiments, the aqueous zinc sulfate is separated from solid tyre char residue by filtration.

In some embodiments, the solid zinc sulfate is recovered by one or more of evaporation, crystallisation and precipitation.

In some embodiments, the solid zinc sulfate is recovered by evaporation of water from aqueous sulfate to reduce the water content, at a temperature in the range of from 50 to 100° C., and subsequent precipitation of solid zinc sulfate.

In some embodiments, the reduction of water content during evaporation is controlled so that subsequent precipitation produces solid zinc sulfate heptahydrate.

In some embodiments, the process comprises the step of calcining solid zinc sulfate.

In some embodiments, the calcining step is carried out at a temperature in the range of from about 400 to about 800° C.

In another aspect, there is provided a process for producing a transition metal-doped zinc sulfate, comprising contacting tyre char with aqueous sulfuric acid and producing a mixture of aqueous zinc sulfate and solid tyre char residue; separating aqueous zinc sulfate from solid tyre char residue; recovering solid zinc sulfate from the aqueous zinc sulfate; contacting zinc sulfate with a salt of a further transition metal; and producing a transition metal-doped solid zinc sulfate.

In some embodiments, the further transition metal is palladium, iron, cobalt or nickel.

In some embodiments, the solid zinc sulfate is recovered from the aqueous zinc sulfate prior to contacting with a salt of a further transition metal salt.

In some embodiments, the solid zinc sulfate is mixed with a salt of a further transition metal in the presence of an organic solvent. In some embodiments, the organic solvent is ethanol.

In some embodiments, the solid zinc sulfate is mixed with the salt of a further transition metal using ultrasonication.

In some embodiments, the solid zinc sulfate is mixed with the salt of a further transition metal present in a solvent-soluble form so as to impregnate the zinc sulfate with the further transition metal.

In some embodiments, the organic solvent is removed by heating and/or vacuum drying.

In some embodiments, the salt of a further transition metal is vaporised and then condensed onto the surface of solid zinc sulfate.

In some embodiments, the salt of a further transition metal is mixed with aqueous zinc sulfate and transition metal-doped solid zinc sulfate is produced by coprecipitation and/or cocrystallization.

In some embodiments, a stabilising and/or acidity-adjusting metal salt is added.

In some embodiments, the stabilising and/or acidity-adjusting metal salt is mixed with aqueous zinc sulfate and with the salt of the further transition metal, and transition metal-doped solid zinc sulfate is produced by coprecipitation and/or co-crystallization.

In some embodiments, the processes according to the invention further comprises the step of calcining the transition metal-doped solid zinc sulfate. In some embodiments, the calcining step is carried out at a temperature in the range of from about 400 to about 800° C.

In some embodiments, following calcination, the transition metal-doped solid zinc sulfate is subjected to a reduction step, by exposure to hydrogen at a temperature in the range of from about 400 to about 800° C.

There is also provided use of a zinc sulfate produced according to a process as defined herein, or use of a transition metal-doped zinc sulfate produced according to a process as defined herein, as a catalyst for producing furfural from lignocellulosic material or a fraction thereof.

In another aspect, there is provided a process for regenerating an active transition metal-doped zinc sulfate catalyst which has been used for producing furfural from lignocellulosic material or a fraction thereof, comprising subjecting the transition metal-doped zinc sulfate to a reduction step, by exposure to hydrogen at a temperature in the range of from about 400 to about 800° C.

In another aspect, there is provided solid zinc sulfate when produced according to processes as defined herein.

In another aspect, there is provided a transition metal-doped solid zinc sulfate, wherein the transition metal is selected from the group consisting of palladium, iron, nickel and cobalt.

In some embodiments, the transition metal-doped solid zinc sulfate contains at least 50 wt % zinc sulfate.

In some embodiments, the transition metal of the transition metal-doped solid is palladium. In further embodiments, the transition metal-doped solid contains palladium metal and palladium oxide.

In some embodiments, the transition metal-doped solid zinc sulfate contains 1 to 5 wt % transition metal.

In some embodiments, the transition metal-doped solid zinc sulfate contains a stabilising and/or acidity-adjusting additional metal.

In another aspect, there is provided a transition metal-doped zinc sulfate produced according to a process as defined herein.

### BRIEF DESCRIPTION OF THE DRAWINGS

FIG. 1 shows a chart showing the selectivity of different products obtained from pyrolysis of D-allose (a) and the convsersion of D-allose with different catalysts at 400° C.

FIG. 2 shows charts showing selectivity of different products obtained from pyrolysis of D-allose and the convsersion of D-allose with (a) and without ZnSO₄/PdO catalyst (b) at different temperatures.

FIG. 3 shows charts showing selectivity of different products obtained from pyrolysis of D-allose (a) and the conversion of D-allose (b) at different catalyst to allose mass ratios at 400° C.

FIG. 4 shows charts showing selectivity of different products obtained from pyrolysis of D-allose (a) and the conversion of D-allose (b) at different water to allose mass ratios with eight times ZnSO₄/PdO as catalyst at 400° C.

FIG. 5 shows charts showing selectivity of different products obtained from pyrolysis of D-glucose and the conversion of D-glucose with different catalysts (a) and different catalyst to glucose mass ratios (b) at 400° C.

FIG. 6 shows DTG analyses of D-glucose (a) and D-allose (b) with and without catalysts at a catalyst to glucose or allose mass ratio of two.

FIG. 7 shows a chart showing selectivilty of different products obtained from pyrolysis of xylan and the conversion of xylan with different catalysts (catalyst to xylan mass ratio: 4) at 400° C.

### DETAILED DESCRIPTION

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “a metal” includes mixture of two or more such metals, and the like.

The present disclosure refers to the entire contents of certain documents being incorporated herein by reference. In the event of any inconsistent teaching between the teaching of the present disclosure and the contents of those documents, the teaching of the present disclosure takes precedence.

### Processes for Producing Furfural

In a first aspect, the present disclosure relates to a process for producing furfural comprising the steps of pyrolyzing a lignocellulosic material or a fraction thereof and producing furfural, wherein the production of furfural is catalysed by a solid zinc sulfate-rich catalyst.

The inventors have found that improved selectivity and/or yield of furfural is observed when zinc sulfate-rich catalysts are used. The processes according to the invention can be carried out with short residence times, therefore enabling high throughput. Additionally, the processes according to the invention utilising zinc sulfate-rich catalysts can generate low levels of wastewater, and can lead to low levels of corrosion to process equipment.

Furfural is an important industrial chemical derivable from biomass which can be used as bio-based starting substrate for the production of various high-value-added chemicals.

Furfural has the chemical structure

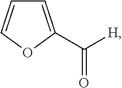

and is also known by the name furan-2-carbaldehyde, furan-2-carboxaldehyde, fural, and 2-furaldehyde. As well as being an important chemical in its own right, furfural can for example be converted into liquid hydrocarbon fuel, into derivative monomers for plastics, or into food additives and pharmaceuticals. Specific examples of downstream chemicals which furfural can be used to produce include furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin.

The process involves the use of lignocellulosic material or a fraction thereof as a feedstock. Lignocellulosic materials contain cellulose, hemicellulose and/or lignin. In some embodiments, the lignocellulosic material used in the process is selected from the group consisting of wood chips, sawdust, sugar cane, corncobs, bagasse, oat hulls, cottonseed hulls, rice hulls and wheat bran. In some embodiments, the lignocellulosic material used in the process is sugar cane. In some embodiments, the lignocellulosic material used in the process is bagasse. In some embodiments, the lignocellulosic material used in the process is corncobs.

If desired, a fraction of a lignocellulosic material may be used, rather than crude lignocellulose. In some embodiments a cellulosic fraction is used (e.g. a fraction which contains at least 50 wt %, 60 wt %, 70 wt %, 80 wt %, 90 wt % or at least 95 wt % cellulosic material), for example cellulose may be used. In some embodiments, a hemicellulosic fraction is used (e.g. a fraction which contains at least 50 wt %, 60 wt %, 70 wt %, 80 wt %, 90 wt % or at least 95 wt % hemicellulosic material), for example hemicellulose may be used. In some embodiments, a fraction which is low in lignin material (e.g. which contains less than 10 wt %, less than 5 wt %, or less than 2 wt % lignin material) or which is free from lignin material is used.

Feedstocks for the furfural production process include monosaccharides, disaccharides, oligosaccharides, polysaccharides, and mixtures thereof. In some embodiments, a lignocellulosic fraction is used which is selected from the group consisting of a monosaccharide, a disaccharide an oligosaccharide and a polysaccharide.

In some embodiments, a monosaccharide is used, or a mixture of monosaccharides. In some embodiments, a disaccharide is used, or a mixture of disaccharides, or a mixture of a monosaccharide and a disaccharide. In some embodiments, an oligosaccharide is used, or a mixture of oligosaccharides, or a mixture of oligosaccharide and monosaccharide, or a mixture of oligosaccharide and disaccharide, or a mixture of oligosaccharide, monosaccharide and disaccharide is used. In some embodiments, a polysaccharide is used, or a mixture of polysaccharides, or a mixture of polysaccharide and monosaccharide, or a mixture of polysaccharide and disaccharide, or a mixture of polysaccharide and oligosaccharide, or a mixture of polysaccharide, oligosaccharide, monosaccharide and/or disaccharide is used.

In some embodiments, a monosaccharide-rich fraction (e.g. a fraction containing at least 50 wt %, at least 60 wt % at least 70 wt %, at least 80 wt %, at least 90 wt % or at least 95 wt % monosaccharide), or disaccharide-rich fraction (e.g. a fraction containing at least 50 wt %, at least 60 wt % at least 70 wt %, at least 80 wt %, at least 90 wt % or at least 95 wt % disaccharide), or oligosaccharide-rich fraction (e.g. a fraction containing at least 50 wt %, at least 60 wt % at least 70 wt %, at least 80 wt %, at least 90 wt % or at least 95 wt % oligosaccharide) may be used.

Specific examples of monosaccharides suitable for use in the processes according to the invention include allose, glucose, xylose, arabinose, mannose, fructose and galactose. In some embodiments, the feedstock used in the process is a monosaccharide or a monosaccharide-rich fraction wherein the monosaccharide is selected from the group consisting of allose, glucose, xylose, arabinose, mannose, fructose and galactose. In some embodiments, the feedstock is allose or an allose-rich fraction (e.g. containing at least 50 wt % allose). In some embodiments, the feedstock is glucose or a glucose-rich fraction (e.g. containing at least 50 wt % glucose).

Specific examples of disaccharides suitable for use in the processes according to the invention include sucrose, lactose and maltose. In some embodiments, the feedstock used in the process is a disaccharide or a disaccharide-rich fraction wherein the disaccharide is selected from the group consisting of sucrose, lactose and maltose.

In some embodiments, an oligosaccharide is used. In some embodiments, a polysaccharide is used. Specific examples of oligosaccharides and/or polysaccharides include xylan, mannan, araban, fructan and cellulose. In some embodiments, the fraction of lignocellulosic material used is xylan. In some embodiments, the fraction of lignocellulosic material used is cellulose.

When a lignocellulosic fraction is used, it may be obtained from lignocellulosic material by any suitable process step or steps. Such process step or steps may if desired be carried out as part of the present process. Alternatively, pre-treatment or pre-processing may be carried out separately from the present process and, e.g. a lignocellulosic fraction used which has been obtained from a supplier.

Where the process includes a pre-processing step or steps, the steps may for example be to carry out one or more of: separation of a cellulosic, hemicellulosic and/or lignin fraction from one or more other fractions of lignocellulosic material; hydrolysis of saccharide linkages (e.g. present in poly- and/or oligosaccharides; and separation of a monosaccharide fraction and/or disaccharide fraction and/or oligosaccharide fraction and/or a polysaccharide fraction.

For example, lignocellulosic material may be exposed to increased temperatures and/or pressures, and/or may be subjected to enzymatic hydrolysis and/or may be subjected to acidic and/or alkali conditions to produce monosaccharides, disaccharides, and/or oligosaccharides.

In some embodiments, one or more pre-processing steps may be undertaken to remove a lignin fraction and/or hydrolyse saccharide linkages. This can be achieved for example by physical pre-treatment such as fragmentation by grinding, chemical pre-treatment such as with acidic or alkaline solutions and solvents, or biological pre-treatment such as with microorganisms including bacteria and or through enzymatic saccharification. For example, if wood chips are used as the feedstock, one or more pre-processing steps may be performed in order to improve the retrieval of cellulosic matter and thus improve the resultant yield of furfural.

If desired, the fraction of interest may be separated from other components to provide a feedstock rich in the material of interest. Examples of suitable techniques include aqueous/organic extraction, precipitation/crystallisation of materials from solution and separation of solid/liquid fractions (e.g. filtration, decanting).

In situations where it is desirable to remove a lignin fraction from a solid lignocellulosic material prior to use in the process, in some embodiments, pre-processing comprises treating a lignocellulosic material with aqueous alkali at elevated temperature to solubilise lignin, and separating the solid and liquid components.

The process for producing furfural comprises pyrolyzing the lignocellulosic material or fraction thereof. Pyrolysis is the thermal decomposition of materials utilising elevated temperature, and is typically carried out in the presence of nitrogen or argon.

Pyrolysis of the lignocellulosic material or fraction thereof can be carried out at any suitable temperature, for example at a temperature in the range of from about 200 to about 600° C. In some embodiments, the pyrolysis step is carried out at a temperature range of from about 300 to about 500° C., or from about 300 to about 400° C., or from about 350 to about 450° C., or from about 400 to about 500° C. In some embodiments, the pyrolysis step is carried out at a temperature of about 300° C., about 350° C., about 400° C., about 450° C. or about 500° C.

The pyrolysis step is carried out for a suitable period of time to effectively pyrolyze the lignocellulosic material or fraction thereof. For example, the pyrolysis step has a duration (e.g. the holding or residence time for the pyrolysis reaction) in the range of from about 5 s to about 2 minutes, or from about 10 s to about 60 s, or from about 20 s to about 50 s. In some embodiments, the duration of the pyrolysis step (the holding time) is in the range of from about 25 s to 45 s. In some embodiments, the duration of the pyrolysis step is about 10 s, about 15 s, about 20 s, about 25 s, about 30 s, about 35 s, about 40 s, about 45 s, about 50 s, about 55 s, or about 60 s.

Any suitable pyrolysis reactor may be used to carry out the pyrolysis step, for example a fluidised bed reactor. In some embodiments, a fluidised bed reactor is used.

The present inventors have discovered that the use of a solid zinc sulfate-rich catalyst exhibits a high catalytic activity that provides high conversion and selectivity for production of furfural when used in catalytic pyrolysis of lignocellulosic material.

As defined herein, the term “zinc sulfate-rich” refers to a material containing at least 20 wt % zinc sulfate. In some embodiments, the zinc sulfate-rich material (e.g. a zinc sulfate-rich catalyst) comprises at least 30 wt %, at least 40 wt %, at least 50 wt %, at least 60 wt %, at least 70 wt %, at least 80 wt %, at least 90 wt % zinc sulfate, or at least 95 wt % zinc sulfate, or at least 98 wt % zinc sulfate, or at least 99 wt % zinc sulfate. In some embodiments, the catalyst consists essentially of, or consists of, zinc sulfate.

In some embodiments, the zinc sulfate may be present in anhydrous or hydrate form. For example, in some embodiments, the zinc sulfate may be zinc sulfate heptahydrate. In some other embodiments, the zinc sulfate may be anhydrous zinc sulfate. References to wt % of zinc sulfate in the present disclosure refer to the wt % of the form of zinc sulfate used. For example, where zinc sulfate heptahydrate is used, the term wt % zinc sulfate refers to the wt % of zinc sulfate heptahydrate present in the material (e.g. present in the zinc-sulfate rich catalyst).

Any suitable amount of solid zinc sulfate-rich catalyst may be used in the process. In some embodiments, the weight ratio of catalyst to lignocellulosic material is in the range of from 1:1 to 20:1, for example in the range of from 2:1 to 15:1, or from about 6:1 to 10:1. In some embodiments, the weight ratio of catalyst to feedstock is about 1:1, about 2:1, about 3:1, about 4:1, about 5:1, about 6:1, about 7:1, about 8:1, about 9:1, about 10:1, about 11:1, about 12:1, about 13:1, about 14:1 or about 15:1.

Different forms of solid zinc sulfate-rich catalyst can be used in the process. For example, transition metal-doped or undoped forms of catalyst may provide particularly advantageous results depending on the lignocellulosic material used as feedstock. Whilst in some embodiments, pure or substantially pure zinc sulfate may be used, in some other embodiments, the solid zinc sulfate-rich catalyst contains a further transition metal. The reference to a further transition metal is to be understood as being a transition metal other than zinc.

As demonstrated by the examples, doping of zinc sulfate with a transition metal such as palladium can provide desirable properties when used in the processes according to the present disclosure. In some embodiments, the further transition metal is selected from the group consisting of palladium, iron, copper, cobalt and nickel. In some embodiments, the further transition metal is palladium or iron. In some embodiments, the further transition metal is palladium.

Without being bound by any particular theory, it is considered that the incorporation of a transition metal such as palladium assists in the cleavage of C—C bonds present in C6 saccharides, thereby improving the conversion and subsequent yield of furfural from the feedstock.

Thus, in some embodiments, a solid zinc sulfate-rich catalyst which has been doped with a further transition metal (e.g. palladium) is used, and the lignocellulosic material or fraction thereof used in the process is one which is rich in C6 saccharides (e.g. C6 monosaccharides, C6 disaccharides, C6 oligosaccharides, and/or C6 polysaccharides). A lignocellulosic material or fraction which is rich in C6 saccharides is one which contains at least 50 wt %, at least 60 wt % at least 70 wt %, at least 80 wt %, at least 90 wt % or at least 95 wt % C6 saccharide.

It has also been found that, for feedstocks rich in C5 saccharides, such as xylan, solid zinc sulfate catalysts which are not doped with a further transition metal perform particularly well in the process. Accordingly, in some embodiments, as solid zinc sulfate-rich catalyst is used which has not been doped with a further transition metal, and the lignocellulosic material or fraction thereof used in the process is one which is rich in C5 saccharides (e.g. C5 monosaccharides, C5 disaccharides, C5 oligosaccharides, and/or C5 polysaccharides). A lignocellulosic material or fraction which is rich in C5 saccharides is one which contains at least 50 wt %, at least 60 wt % at least 70 wt %, at least 80 wt %, at least 90 wt % or at least 95 wt % C5 saccharide.

Where the solid zinc sulfate-rich catalyst is doped with a further transition metal, the further transition metal is typically present in an amount of up to 20 wt %. In some embodiments, the transition metal is present in an amount of from about 0.1 to about 10 wt %, or from about 1 to about 5 wt % based on the total weight of the catalyst. In some embodiments, the transition metal is present in an amount of about 0.5 wt %, about 1 wt %, about 1.5 wt %, about 2 wt %, about 2.5 wt %, about 3 wt %, about 3.5 wt %, about 4 wt %, about 4.5 wt % or about 5 wt %.

In some embodiments, the solid zinc sulfate-rich catalyst contains from about 0.1 to about 5 wt % further transition metal, for example from 0.2 to 5 wt %, or from 0.5 to 3 wt %, or about 0.2 wt %, about 0.5 wt %, about 1 wt %, about 2 wt %, about 3 wt %, about 4 wt %, or about 5 wt % further transition metal.

Where the solid zinc sulfate-rich catalyst is doped with a further transition metal, the further transition metal may for example be mixed with the solid zinc sulfate-rich catalyst, or co-precipitated. Alternatively, the surface (or at least part of the surface) of the solid zinc sulfate-rich catalyst may be coated with further transition metal.

Any suitable form of the further transition metal may be used with the solid zinc sulfate-rich catalyst. For example, a salt form of the further transition metal may be used. The further transition metal may also or instead be present as an oxide. The further transition metal may also or instead be present in the form of the metal itself. In some embodiments, the further transition metal may be present in a mixture of forms, for example as an oxide and as the metal itself. For example, in some embodiments, the further transition metal present on the surface of the solid zinc sulfate-rich catalyst may be present predominantly as an oxide and further transition metal present in the interior of the zinc sulfate-rich catalyst may be present predominantly as the metal. In the case of palladium, for example, in some embodiments palladium may be present in the solid zinc sulfate-rich catalyst as palladium metal and/or palladium oxide.

Where the further transition metal is added using a salt form of the further transition metal, exemplary salts include nitrates, sulfates, acetates, chlorides, bromides and iodides. In some embodiments, further transition metal is added using a nitrate salt of the transition metal. In some embodiments, the further transition metal is palladium, and palladium nitrate is added to solid zinc sulfate.

In some embodiments, the transition metal-doped solid zinc sulfate-rich catalyst contains a stabilising and/or acidity-adjusting additional metal.

The catalyst may be subjected to additional processing steps prior to use if desired. For example, the catalyst be subjected to granulation, to produce granules of catalyst.

The process may be carried out using any suitable apparatus or reactor equipment. In some embodiments, the process is carried out as a batch process. In some other embodiments, the process is carried out as a continuous or semi-continuous process.

Pyrolysis of the lignocellulosic material or fraction thereof, and production of furfural catalysed by solid zinc sulfate-rich catalyst, may in some embodiments be carried out as a single step in a single reactor. However, in other embodiments, pyrolysis of the lignocellulosic material or fraction thereof and production of furfural catalysed by solid zinc sulfate-rich catalyst may be carried out as separate steps. For example, pyrolysis may be carried out to produce a lignocellulosic vapour stream (e.g. in a first reactor), and the lignocellulosic vapour stream then contacted with solid zinc sulfate-rich catalyst and producing furfural (e.g. in a second reactor). For example, a lignocellulosic vapour stream may be passed over and/or through a bed of solid zinc sulfate-rich catalyst. In some embodiments, the step of producing furfural is carried out using a moving bed reactor, or using a packed bed reactor. For example, a reactor (e.g. moving bed reactor or packed bed reactor) containing granulated catalyst may be used.

In some embodiments, pyrolysis and contacting with catalyst are carried out as separate steps, with the lignocellulosic material or fraction thereof being pyrolyzed in a first step (for example in a fluidised bed reactor) to produce a lignocellulosic vapour stream, which is carried by a stream containing argon and/or nitrogen to a second reactor (e.g. a packed bed reactor or moving bed reactor) containing granulated catalyst, with water vapour being introduced if desired, and the lignocellulosic vapour stream then contacted with catalyst and furfural being produced, resulting in a product vapour stream containing furfural.

Production of furfural can be carried out at any suitable temperature, for example at a temperature in the range of from about 200 to about 600° C. In some embodiments, the production of furfural is carried out at a temperature range of from about 300 to about 500° C., or from about 300 to about 400° C., or from about 350 to about 450° C., or from about 400 to about 500° C. In some embodiments, the production of furfural is carried out at a temperature of about 300° C., about 350° C., about 400° C., about 450° C. or about 500° C.

Production of furfural by contacting with the solid zinc sulfate-rich catalyst is carried out for a suitable period of time to provide high conversion to furfural. For example, contacting with the catalyst may be carried out for a period in the range of from about 5 s to about 2 minutes, or from about 10 s to about 60 s, or from about 20 s to about 50 s. In some embodiments, contacting with the solid zinc sulfate-rich catalyst is carried out for a period in the range of from about 25 s to 45 s. In some embodiments, the duration is about 10 s, about 15 s, about 20 s, about 25 s, about 30 s, about 35 s, about 40 s, about 45 s, about 50 s, about 55 s, or about 60 s.

Where pyrolysis and production of furfural are carried out as part of a single step, the above-listed temperature and time periods may for example apply to that single step.

The inventors have found that the presence or absence of water vapour/steam can influence the eventual yield of furfural. In some embodiments the process for producing furfural is conducted in the absence of steam, for example where the production of furfural is carried out under nitrogen or argon atmosphere. In some other embodiments, the production of furfural is carried out in the presence of steam, for example where steam/water vapour is added to the nitrogen and/or argon atmosphere. In some embodiments, the weight ratio of steam to lignocellulosic material or fraction thereof used is in the range of from about 0.1:1 to about 30:1, or from about 5:1 to about 30:1, or from about 0.1:1 to about 20:1, or from about 1:1 to about 20:1, or from about 8:1 to about 20:1. In some embodiments, the weight ratio of steam to lignocellulosic material is about 0.1:1, about 0.5:1, about 1:1, about 2:1, about 3:1, about 4:1, 5:1, about 6:1, about 7:1, about 8:1, about 9:1, about 10:1, about 11:1, about 12:1, about 13:1, about 14:1, about 15:1, about 16:1, about 17:1 about 18:1, about 19:1, or about 20:1.

Typically following the reaction, a product vapour stream containing furfural is obtained which is readily separable from the solid zinc sulfate-rich catalyst.

If desired, the product mixture containing furfural may be separated from other organic components. For example, amounts of side products such as a light compound (LC, for example, acetic acid and furan), levulinic acid (LA), 5-hydroxymethylfurfural (5-HMF) and levoglucosenone (LGO) may be produced, and it may be desirable to separate the furfural from those components, or to decrease the levels of those side-products. Any suitable processing technique may be used to separate and/or purify furfural.

In some embodiments, furfural is separated from other components by distillation. For example, fractional distillation may be used, e.g. using a series of columns and appropriate temperature conditions so as to separate furfural from high and/or low boiling components of the product mixture as required.

In some other embodiments, furfural is separated by selective condensation of a product vapor stream containing furfural. The product vapor stream containing furfural is present at elevated temperature. By adjusting (i.e. reducing) the temperature of the product stream appropriately, byproducts having a higher boiling point than furfural can first be separated from furfural which remains in the gaseous phase. Following separation of high boiling materials, the furfural-containing vapour stream can then be subjected to a lower temperature (e.g. below the boiling point of furfural), resulting in condensation of furfural which can be separated from byproducts having a lower boiling point.

As discussed above, the present process produces furfural. Accordingly, there is also provided furfural produced by a process as defined herein.

The process for producing furfural also provides access to downstream chemicals which can be produced from furfural. Accordingly, in another aspect, there is provided a process for producing one or more of furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin, comprising producing furfural in accordance with processes according to the invention; and converting the furfural into furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin.

Methods of producing the above-referenced compounds from furfural are known in the art. For example, furfuryl alcohol may be produced, for example by reduction using hydrogenation and a suitable catalyst (such as a nickel, palladium, platinum, or zinc-copper-based catalyst), electrocatalytic reduction, enzymatic reduction, or biotransformation using a suitable bacteria or yeast (see for example U.S. Pat. No. 2,077,409, Gutierrez et al, Appl. Biochem. Biotechnol., 2002, 98-100, 327-40; Brosnahan et al, Nanoscale, 2021, 13,2312-2316, the entire contents of which are incorporated herein by reference).

Furoic acid may for example be produced by oxidation of furfural, or furfuryl alcohol (e.g. with permanganate or dichromate). As a further example, an aqueous Cannizaro reaction may be used (e.g. treating furfural with aqueous alkali such as sodium hydroxide) to produce a mixture of furoic acid (following acidification of the product mixture) and furfuryl alcohol. Furan may for example be produced by decarbonylation of furfural (e.g. over a palladium, zeolite or nickel-magnesium oxide catalyst), or by decarboxylation of furoic acid (which may itself be produced from furfural). See for example, Jiminez-Gomez et al, ACS Sustainable Chem. Eng., 2019, 7, 8, 7676-7685, WO2015/020845, EP3126342, and U.S. Pat. No. 7,044,480, the entire contents of which are incorporated herein by reference.

Tetrahydrofuran may for example be produced by hydrogenation of furan (which can itself be produced from furfural) e.g. using a palladium catalyst such as palladium oxide, or directly from furfural without isolation of an intermediate product (e.g. by reduction using hydrogen and a palladium catalyst). See for example WO2014/118806, and Org. Synth. 1936, 16, 77, the entire contents of which are incorporated herein by reference.

Levulinic acid may for example be produced by treatment with an alcohol (e.g. methanol) and dialkoxymethane (e.g. dimethoxymethane) to produce alkyl levulinate, which can then be hydrolysed (e.g. under acidic aqueous conditions) to produce levulinic acid (see for example Shao et al, Green Energy and Environment, 2019, 4, 4, 400-413, the entire contents of which are incorporated herein by reference).

Butadiene may for example be produced by conversion of furfural to tetrahydrofuran, for example as discussed above, and conversion of tetrahydrofuran to butadiene, for example by dehydration-ring-opening (e.g. using a zeolite catalyst, such as a silica-phosphorous zeolite (see e.g. Abdelrahman et al, ACS Sustainable Chem. Eng., 2017, 5, 5, 3732-3736, the entire contents of which are incorporated herein by reference).

Hexamethylenediamine may for example be produced by conversion of furfural to 1,6-hexanediol, followed by amination to produce hexamethylene diamine (see e.g. U.S. Pat. No. 9,518,005, the entire contents of which are incorporated herein by reference).

Tetrahydrofurfuryl alcohol may for example be produced from furfuryl alcohol (which may itself be produced from furfural), or produced directly from furfural, for example by hydrogenated in the presence of a suitable catalyst, such as a hydroxyapatite-supported palladium catalyst (see e.g. Li et al, Ind. Eng. Chem. Res., 2017, 56, 31, 8843-8849, the entire contents of which are incorporated herein by reference).

2-Methyltetrahydrofuran may for example be produced by reduction of furfural, for example by hydrogenation in the presence of a suitable catalyst (e.g. reduction in the presence of a Co-based catalyst to produce 2-methylfuran, and then reduction in the presence of a Ni-based catalyst to produce 2-methyltetrahydrofuran). See for example Liu et al, Molecular Catalysis, 2020, 490, 110951, the entire contents of which are incorporated herein by reference.

Furfural-phenolic resin may for example be produced by reaction of furfural with a phenol, for example in the presence of a base such as sodium hydroxide.

### Processes for Producing Solid Zinc Sulfate

The disposal of rubber tyres at the end of their life cycle presents challenges in waste management to many developing and developed countries. Improper disposal of waste or scrap tyres can result in numerous health, safety and environmental hazards. Waste tyre pyrolysis is a process that is often used to process waste tyres, involving exposure to high temperatures in an oxygen-poor atmosphere. Typically, pyrolysis of waste tires yields a significant component of solid tyre char, but there is a limited demand for the post-pyrolytic char since it typically contains numerous contaminants. One use for post-pyrolytic char is to convert it to active carbon, however such processes often result in leaching of metal ions into water, posing an environmental hazard.

The inventors have discovered a process which enables production of zinc sulfate from waste sources such as tyre char, for example facilitating the production of solid zinc sulfate-rich catalysts as described above. Accordingly, in another aspect, there is provided a process for producing solid zinc sulfate comprising the steps of contacting tyre char with aqueous sulfuric acid and producing a mixture of aqueous zinc sulfate and solid tyre char residue; separating aqueous zinc sulfate from solid tyre char residue; and recovering solid zinc sulfate from the aqueous zinc sulfate.

Utilising the present process enables waste zinc to be captured and isolated in a solid state and used in materials, or downstream processes, such as in the pyrolysis and conversion of lignocellulosic materials into furfural.

The process involves contacting the tyre char with an aqueous sulfuric acid. Typically, aqueous sulfuric acid and tyre char are admixed (e.g. tyre char may be added to aqueous sulfuric acid, or vice versa), and then mixed for a suitable period of time to allow leaching of zinc from the tyre residue and production of aqueous zinc sulfate. The mixture may for example be agitated, or mixed using a mixer, to facilitate the reaction. In some embodiments, the mixing of the tyre char and sulfuric acid is conducted under controlled stirring at a specified rate. Where the mixture is stirred, the stirring rate may for example be at a rate in the range of from about 100 rpm to about 600 rpm, or from about 200 rpm to about 400 rpm, or about 100 rpm, about 200 rpm, about 300 rpm, about 400 rpm, about 500 rpm, or about 600 rpm.

The step of contacting tyre char with aqueous sulfuric acid is carried out at a temperature suitable to leach zinc from the tyre char and produce aqueous zinc sulfate. For example, the step may be conducted at a temperature in the range of from about 25 to about 90° C., or from about 40 to about 80° C., or at about 30° C., about 40° C., about 50° C., about 60° C., about 70° C., about 75° C., about 80° C., about 85° C., or about 90° C.

The concentration of sulfuric acid in the aqueous sulfuric acid is typically in the range of from about 0.5 to about 15 moles per litre, for example in some embodiments it is in the range of from about 1 to about 10 moles per litre, or from about 1 to about 5 moles per litre. In some embodiments the aqueous sulfuric acid concentration is about 1, about 2, about 3, about 4, or about 5 moles per litre.

An amount of aqueous sulfuric acid is used so as to facilitate extraction of zinc from tyre char. The volume:mass ratio of aqueous sulfuric acid to tyre char is typically in the range of from about 1:1 to 20:1, or from about 1:1 to 10:1, or from about 1.5:1 to 10:1, or from about 1:1 to 6:1, or from about 1.5:1 to 6:1, or about 1:1 or about 2:1 or about 3:1, or about 4:1, or about 5:1, or about 6:1.

In some embodiments, the aqueous sulfuric acid has a sulfuric acid concentration in the range of from about 1 to about 5 moles per litre and is used at a volume:mass ratio in the range of from 1.5:1 to 10:1 aqueous sulfuric acid to tyre char.

The step of contacting the tyre char with aqueous sulfuric acid is carried out for a suitable period of time to facilitate extraction of zinc as zinc sulfate. In some embodiments, the contacting step is carried out for a period in the range of from 5 minutes to 24 hours, for example for a period in the range of from 5 minutes to 12 hours, or from 5 minutes to 6 hours, or from 10 minutes to 2 hours, or about 10 minutes, about 20 minutes, about 30 minutes, about 40 minutes, about 50 minutes, about 1 hour, about 1.5 hours, or about 2 hours.

The contacting of tyre char with aqueous sulfuric acid may be carried out using any suitable equipment. For example, a continuous stirred tank reactor (CSTR) may be used.

If desired, the tyre char may undergo one or more pre-processing steps prior to contacting with aqueous sulfuric acid. For example, following pyrolysis of scrap tyres, the tyre char produced may if desired be ground and sieved to reduce particle size, for example to produce tyre char having a mean particle size in the range of from 100 to 500 μm, or from about 200 to 400 μm, or from about 250 to 300 μm. Decreasing particle size is understood to assist in facilitating extraction of zinc. Steel and/or wire components may for example be removed from the tyre char prior to contacting to aqueous sulfuric acid. The tyre char may for example be washed, e.g. with water or an aqueous solvent, prior to contacting with aqueous sulfuric acid.

Accordingly, in some embodiments, prior to contacting with aqueous sulfuric acid, tyre char is subjected to one or more of the following process steps:
reduction of particle size (e.g. by grinding the tyre char and/or sieving);
removal of steel and/or wire from the tyre char; and
washing wish water or an aqueous solvent.

Following the step of contacting tyre char with is step, the aqueous zinc sulfate is separated from the solid tyre char residue. Numerous methods may be used to separate the aqueous zinc sulfate from solid tyre char residue, for example by filtration, or by allowing the solid portion to settle and decanting, siphoning off or otherwise collecting the liquid component.

In some embodiments, the aqueous zinc sulfate is separated from solid tyre char residue by filtration. If desired, the solid tyre char residue may for example be washed with water or an aqueous solvent to recover additional zinc sulfate.

Following separation from tyre char residue, solid zinc sulfate is recovered from the aqueous zinc sulfate. Suitable processing techniques include recovering solid zinc sulfate by one or more of evaporation, crystallisation and precipitation.

In some embodiments, water is removed by one or more of evaporation and or distillation. The aqueous zinc sulfate may for example be heated to a temperature in the range of from 50 to 100° C., e.g. at about 50° C., about 60° C., about 70° C., about 80° C., about 90° C., about 95° C., or about 100° C. Vacuum distillation may for example be used, so that water can be removed at lower temperatures than at atmospheric pressure.

For example, in some embodiments, water is evaporated from aqueous zinc sulfate by boiling the mixture (e.g. heating to about 100° C.). In some other embodiments, the pressure may be reduced and water evaporated by heating at a temperature in the range of from 50 to 60° C.

The effect of evaporation and/or distillation is to concentrate the aqueous zinc sulfate mixture. Typically, the water level is reduced such that a saturated solution of zinc sulfate is produced, and solid zinc sulfate crystallises and/or precipitates from solution.

Accordingly, in some embodiments, solid zinc sulfate is recovered by evaporation of water from aqueous sulfate to reduce the water content, at a temperature in the range of from 50 to 100° C., and subsequent precipitation and/or crystallisation of solid zinc sulfate.

In some other embodiments, solid zinc sulfate may be obtained by addition of an antisolvent, for example a water-miscible organic solvent, resulting in precipitation and/or crystallisation of solid zinc sulfate.

Any suitable equipment may be used to perform evaporation, distillation, precipitation and/or crystallisation steps. For example a crystalliser may be used to obtain solid zinc sulfate, such as for example a Swenson DTE crystalliser.

The solid zinc sulfate obtained can be separated from the water or aqueous solvent by any suitable method, for example by filtration, or by settling the solid component and removing the liquid fraction, e.g. by siphoning or decanting.

In some embodiments, the residual liquid component may be subjected to further processing to recover additional zinc sulfate from solution. For example, in some embodiments, the resultant solution may be subjected to one or more evaporation-precipitation cycles.

In some embodiments, zinc sulfate is recovered from the aqueous zinc sulfate in the form of zinc sulfate heptahydrate.

Accordingly, in some embodiments, an evaporation or distillation step is carried out to remove water and concentrate the zinc sulfate in solution to a concentration such that subsequent crystallisation and/or precipitation produces zinc sulfate heptahydrate. In some embodiments, reduction of water content during evaporation is controlled so that subsequent precipitation produces solid zinc sulfate heptahydrate. In some embodiments, when solid zinc sulfate is recovered by crystallization from water or an aqueous solvent, and the amount of water used is controlled so that crystallization produces solid zinc sulfate heptahydrate.

The evaporation step may for example be controlled under specific conditions so that subsequent precipitation steps produce solid zinc sulfate heptahydrate. For example, an aqueous zinc sulfate mixture may first be heated to about 100° C. and water removed to produce a saturated solution of zinc sulfate, which may subsequently be heated at a lower temperature (e.g. in the range of from 50 to 60° C.) whilst subjecting to reduced pressure, with removal of additional water, to produce zinc sulfate heptahydrate.

Depending on the intended use for the recovered solid zinc sulfate, it may subsequently be exposed to a calcination step, for example to remove impurities. Accordingly, in some embodiments, the process comprises the step of calcining the solid zinc sulfate.

Calcination refers to the process of heating a solid to high temperatures in the absence of air or oxygen. Any suitable equipment allowing for controlled calcination conditions may be used, for example a reactor such as a packed bed reactor.

The calcination step is carried out at elevated temperature, for example in the range of form about 300 to about 1000° C. In some embodiments, the calcination step is carried out at a temperature in the range of from about 400 to about 800° C., for example about 400° C., about 500° C., about 600° C., about 700° C., or about 800° C.

The calcination step may for example be performed in the presence of an inert atmosphere, such as argon or nitrogen.

Upon recovery of zinc from scrap tyre char, the resultant tyre char can subsequently be used as a clean fuel in the power generation plants, or further upgraded into advanced adsorbents such as carbon black using conventional processes. Accordingly, in some embodiments, the process comprises recovering solid tyre char residue. In some embodiments, the process comprises recovering solid tyre char residue and converting the solid tyre char residue into carbon black.

### Processes for Producing Transition Metal-Doped Zinc Sulfate

Also provided herein are processes for producing transition metal-doped zinc sulfate (i.e. zinc sulfate doped with another transition metal which is not zinc). Such materials find use for example as catalysts for producing furfural as discussed herein.

The process comprises contacting tyre char with aqueous sulfuric acid and producing a mixture of aqueous zinc sulfate and solid tyre char residue; separating aqueous zinc sulfate from solid tyre char residue; recovering solid zinc sulfate from aqueous zinc sulfate; contacting zinc sulfate with a salt of a further transition metal; and producing a transition metal-doped solid zinc sulfate.

The steps of contacting tyre char with aqueous sulfuric acid, separating aqueous zinc sulfate from solid tyre char residue, and recovering solid zinc sulfate from aqueous zinc sulfate, may be carried out as described above in the context of discussing processes for producing solid zinc sulfate, and embodiments discussed in relation to those processes also apply to processes for producing transition metal-doped zinc sulfate.

The transition metal with which the zinc sulfate is doped may be any desired transition metal other than zinc. Where the transition metal-doped zinc sulfate is intended for use as catalyst for producing furfural, the transition metal may for example be one which improves catalytic activity, for example it may be selected from the group consisting of palladium, iron cobalt and nickel. In some embodiments, the transition metal is palladium or iron. In some embodiments, the transition metal is palladium.

The transition metal-doped solid zinc sulfate may be produced in a number of ways.

In some embodiments, solid zinc sulfate is recovered from the aqueous zinc sulfate prior to contacting with a salt of a transition metal.

Where the transition metal is added using a salt form of the transition metal, exemplary salts include nitrates, sulfates, acetates, chlorides, bromides and iodides. In some embodiments, transition metal is added using a nitrate salt of the transition metal. In some embodiments, the transition metal is palladium, and palladium nitrate is added to solid zinc sulfate

In embodiments where a salt form of the transition metal is contacted with solid zinc sulfate, this may for example be done by mixing solid zinc sulfate with a transition metal salt in the presence of an organic solvent. Any organic solvent which solubilises the transition metal salt may be used, for example an alcohol solvent may be used. In some embodiments, the organic solvent is ethanol.

Typically, the solid zinc sulfate and solution of transition metal salt are mixed together in a suitable amount. In some embodiments, the liquid to solid ratio is 10 mL/g.

The solution containing the salt of the transition metal and the solid zinc sulfate are typically mixed together for a period of time suitable to ensure good mixing of the components and formation of a homogeneous mixture. Mixing may be accomplished using any suitable means, for example an overhead stirrer may be used. In some embodiments, solid zinc sulfate is mixed with the salt of a further transition metal salt using ultrasonication. Ultrasonication is the use of sound energy at ultrasonic frequencies to agitate and mix particles in a solution. The ultrasonication step may be conducted for a suitable period of time. In some embodiments, the solution containing the solid zinc sulfate catalyst and the salt of a further transition metal are subjected to ultrasonication for a period in the range of from about 5 minutes to 1 hour, or from about 10 to about 30 minutes.

In some embodiments, the solid zinc sulfate is mixed with the salt of a further transition metal salt present in a solvent-soluble form so as to impregnate the zinc sulfate with the further transition metal.

The mixing of solid zinc sulfate with the salt of a further transition metal may be carried out using any suitable equipment. For example, a continuous stirred tank reactor (CSTR) may be used.

Where the transition metal is introduced by means of an organic solvent solution, following mixing, the organic solvent will typically be removed. This may be achieved by for, example, a heating and/or vacuum drying step in order to remove the organic solvent. For example, the mixture may be heated in an oven or autoclave, for example at a temperature in the range of from about 50° C. to about 250° C., or from about 150° C. to about 250° C., or from about 160° C. to about 200, or about 150° C., or about 160° C., or about 170° C., or about 180° C., or about 190° C., or about 200° C., or about 210° C., or about 220° C., or about 230° C., or about 240° C., or about 250° C. Suitable time periods for removal of organic solvent at high temperature may for example be in the range of from 3 to 24 hours, or from 6 to 24 hours, or from 12 to 16 hours, or about 6, about 8, about 10, about 12, about 14, or about 16 hours. In some embodiments, organic solvent is removed by hydrothermal treatment in an autoclave at a temperature in the range of from 150° C. to about 250° C., and for a period in the range of from 12 hours to about 16 hours.

In some embodiments, when solid zinc sulfate is recovered from the aqueous zinc sulfate prior to contacting with a salt of a transition metal, transition metal-doped solid zinc sulfate is produced by condensing transition metal vapor onto the surface of solid zinc sulfate. For example, the salt of a further transition metal may be vaporised and then condensed onto the surface of solid zinc sulfate. This allows direct loading of the transition metal onto the surface of the solid zinc sulfate catalyst.

As an alternative approach, the transition metal-doped solid zinc sulfate may for example be produced by mixing a salt of the transition metal with aqueous zinc sulfate, and transition metal-doped solid zinc sulfate being produced by coprecipitation and/or cocrystallization.

For example, a solution of a transition metal salt in an organic solvent (e.g. an alcohol such as ethanol), may be added to aqueous zinc sulfate to produce a solution, and the mixture then subjected to one or more evaporation, distillation, precipitation and/or crystallisation steps.

Any suitable equipment may be used to perform evaporation, distillation, precipitation and/or crystallisation steps. For example a crystalliser may be used to obtain transition metal-doped solid zinc sulfate, such as for example a Swenson DTE crystalliser.

Any suitable form of the transition metal may be used with the solid zinc sulfate. For example, a salt form of the transition metal may be used. The transition metal may also or instead be present as an oxide. The transition metal may also or instead be present in the form of the metal itself. In some embodiments, the transition metal may be present in a mixture of forms, for example as an oxide and as the metal itself. For example, in some embodiments, the transition metal present on the surface of the solid zinc sulfate may be present as an oxide (or predominantly as an oxide) and transition metal present in the interior of the zinc sulfate may be present as the metal (or predominantly as the metal). In the case of palladium, for example, in some embodiments palladium may be present in and/or on the solid zinc sulfate as palladium metal and/or palladium oxide.

In some embodiments, in addition to incorporating a transition metal such as palladium, iron, cobalt or nickel, an amount stabilising and/or acidity adjusting metal salt is added. Such a step may be conducted in order to improve the efficiency of the doping process, for example by stabilise the transition metal by forming spinels or alloys which are stable and less prone to agglomerate upon thermal treatment. Examples of stabilising and/or acidity adjusting metals which may be used include lanthanum and caesium.

In some embodiments, the stabilising and/or acidity adjusting metal salt is introduced by impregnation on the solid zinc sulfate support, or by co-precipitation with zinc sulfate and transition metal.

In some embodiments, solid zinc sulfate is recovered from the aqueous zinc sulfate prior to mixing with the salt of the transition metal and with the stabilising and/or acidity-adjusting metal salt.

In some embodiments, the stabilising and/or acidity-adjusting metal salt is mixed with aqueous zinc sulfate and with the salt of the transition metal, and transition metal-doped solid zinc sulfate is produced by coprecipitation and/or cocrystallization.

In some embodiments, a calcining step is carried out following production of the transition metal-doped solid zinc sulfate, for example to remove impurities. Accordingly, in some embodiments, the process comprises the step of calcining the transition metal-doped solid zinc sulfate.

A calcination step may serve to provide any one of more of the following benefits: mechanically and thermodynamically stabilize the microstructure of the transition metal-doped zinc sulfate, removing other impurities or volatile substances, providing a more uniform and homogenous distribution of the transition metal-doped zinc sulfate, and/or improving catalytic activity.

Any suitable equipment allowing for controlled calcination conditions may be used, for example a reactor such as a packed bed reactor may be used.

The calcination step is carried out at elevated temperature, for example in the range of form about 300 to about 1000° C. In some embodiments, the calcination step is carried out at a temperature in the range of from about 400 to about 800° C., for example about 400° C., about 500° C., about 600° C., about 700° C., or about 800° C.

The calcination step may for example be performed in the presence of an inert atmosphere, such as argon or nitrogen.

In some embodiments, following calcination, the transition metal-doped solid zinc sulfate is subjected to a reduction step, by exposure to hydrogen at a temperature in the range of from about 400 to about 800° C. for example at a temperature in the range of from about 500 to 700° C., or at about 400° C., or about 500° C., or about 600° C., or about 700° C., or about 800° C. It is considered that carrying out a reduction step may assist in producing a particularly active catalyst. Typically, the reduction step is done in the presence of an inert gas such as hydrogen.

The reduction step may be carried out using any suitable equipment. For example a packed bed reactor may be used.

The zinc sulfate and transition metal-doped solid zinc sulfate produced by the processes described above find use, for example, as catalysts for the production of furfural. Accordingly, there is also provided use of a zinc sulfate produced by a process as defined herein, or of a transition metal-doped solid zinc sulfate produced by a process as defined herein, as a catalyst for producing furfural from lignocellulosic material or a fraction thereof.

Where a transition metal-doped solid zinc sulfate is used as a catalyst for producing furfural, in some embodiments it may be used only once or for a small number of reactions. However, in other embodiments, the catalyst may be used for producing many batches of furfural, or in a continuous process for a prolonged period of time. In some cases, it may be desirable to treat the catalyst after it has been used a number of times or for a sufficient period of time, to regenerate and/or improve activity, for example by subjecting it to reducing conditions as discussed above.

Accordingly, there is provided a process for regenerating an active transition metal-doped zinc sulfate catalyst which has been used for producing furfural from lignocellulosic material or a fraction thereof, comprising subjecting the transition metal-doped zinc sulfate to a reduction step, for example by exposure to hydrogen at a temperature in the range of from about 400 to about 800° C.

As discussed above in relation to processes for producing solid zinc sulfate, the tyre char residue can subsequently be used as a fuel in power plants, or converted into adsorbents such as carbon black using conventional processes. Accordingly, in some embodiments, the process comprises recovering solid tyre char residue. In some embodiments, the process comprises recovering solid tyre char residue and converting the solid tyre char residue into carbon black.

### Zinc Sulfate-Rich Materials and Transition-Metal Doped Zinc Sulfate

The present disclosure also relates to zinc sulfate-rich materials and catalysts themselves, which find use as catalysts in the production of furfural.

Accordingly, in a further aspect there is provided solid zinc sulfate produced by a process as defined herein.

There is also provided a transition metal-doped solid zinc sulfate, wherein the transition metal is selected from the group consisting of palladium, iron, nickel and cobalt. In some embodiments, the transition metal is palladium.

In some embodiments, the transition metal-doped solid zinc sulfate comprises at least 20 wt % zinc sulfate. In some embodiments, the transition metal-doped solid zinc sulfate comprises at least 30 wt %, at least 40 wt %, at least 50 wt %, at least 60 wt %, at least 70 wt %, at least 80 wt %, at least 90 wt % zinc sulfate, or at least 95 wt % zinc sulfate, or at least 98 wt % zinc sulfate, or at least 99 wt % zinc sulfate.

In some embodiments, the zinc sulfate may be present in anhydrous or hydrate form. For example, in some embodiments, the zinc sulfate may be zinc sulfate heptahydrate. In some other embodiments, the zinc sulfate may be anhydrous zinc sulfate. References to wt % of zinc sulfate in the present disclosure refer to the wt % of the form of zinc sulfate used. For example, where zinc sulfate heptahydrate is used, the term wt % zinc sulfate refers to the wt % of zinc sulfate heptahydrate present in the material (e.g. present in the zinc-sulfate rich catalyst).

In some embodiments, the transition metal is selected from the group consisting of palladium, iron, copper, cobalt and nickel. In some embodiments, the transition metal is palladium or iron. In some embodiments, the transition metal is palladium.

The transition metal is typically present in an amount of up to 20 wt %. In some embodiments, the transition metal is present in an amount of from about 0.1 to about 10 wt %, or from about 1 to about 5 wt % based on the total weight of the catalyst. In some embodiments, the transition metal is present in an amount of about 0.5 wt %, about 1 wt %, about 1.5 wt %, about 2 wt %, about 2.5 wt %, about 3 wt %, about 3.5 wt %, about 4 wt %, about 4.5 wt % or about 5 wt %.

The transition metal may for example be mixed with solid zinc sulfate, or co-precipitated. Alternatively, the surface (or at least part of the surface) of solid zinc sulfate may be coated with transition metal.

Any suitable form of the transition metal may be used with the solid zinc sulfate. For example, a salt form of the transition metal may be used. The transition metal may also or instead be present as an oxide. The transition metal may also or instead be present in the form of the metal itself. In some embodiments, the transition metal may be present in a mixture of forms, for example as an oxide and as the metal itself. For example, in some embodiments, the transition metal present on the surface of the solid zinc sulfate may be present as an oxide (or predominantly as an oxide) and transition metal present in the interior of the zinc sulfate-rich catalyst may be present as the metal (or predominantly as the metal). In the case of palladium, for example, in some embodiments palladium may be present in and/or on the solid zinc sulfate as palladium metal and/or palladium oxide.

Where the transition metal is added using a salt form of the transition metal, exemplary salts include nitrates, sulfates, acetates, chlorides, bromides and iodides. In some embodiments, transition metal is added using a nitrate salt of the transition metal. In some embodiments, the transition metal is palladium, and palladium nitrate is added to solid zinc sulfate.

In some embodiments, the transition metal-doped solid zinc sulfate contains a stabilising and/or acidity-adjusting additional metal. The role of the stabilising and/or acidity adjusting additional metal may for example be to improve catalytic performance, or to stabilise the transition metal dopant present on the catalyst by forming spinels or alloys, which may make the catalyst less prone to agglomerate upon thermal treatment.

There is also provided herein a transition metal-doped solid zinc sulfate produced according to a process as defined herein.

### Examples

The present disclosure is further illustrated by the following non-limiting examples.

### Experimental

### 1. Preparation of Zinc Sulfate from Scrap Tyre Char

Scrap tyre char was derived from the pyrolysis of waste scrap tyre (mixture) at 800° C. in a pilot-scale moving bed reactor. The tyre char samples were ground and sieved to 250-300 μm in size and dried at 105° C. for 15 h. For the tyre char, the steel and wires within it were removed prior washing. The elemental analysis (see Table 1) of scrap tyre char was presented in our previous study [17]. The ash content is 13.3 wt % with Zn as the most abundant element followed silicon (Si). Their compositions are 30% and 12 wt % respectively in ash. Other main metals are calcium (Ca, 4.7 wt % on the basis of ash), aluminium (Al, 1.2 wt % on the basis of ash) and iron (Fe, 1.0 wt % on the basis of ash).

To recover the zinc in the tyre char, different concentrations (1-5 mol/L) of sulfuric acid were mixed with the water-washed tyre char with a L/S ratio of 1 mL/g-6 mL/g. All the leaching experiments were carried out in a 250 mL conical flask in a thermostatically controlled water bath at 25° C.-80° C. and stirred for a period of 10 min-120 min. The stirring rate was controlled at 300 rpm for all the cases studied here. subsequently, the slurry was separated by filtration using a filter paper with a 450 μm cut-off size. S4) the Zn-rich leachate was then evaporated at around 100° C. until saturated. The resultant saturated solution was transferred to a water-bath for the zinc sulfate to precipitate out. The resultant solution was further subjected to evaporation-precipitation cycle for three times. Alternatively, the saturated solution was evaporated slowly at 50-60° C. in a low pressure and the resultant crystal precipitate was separated out as ZnSO₄·7H₂O.

TABLE 1
Elemental composition of scrap tyre char

| | Ash | Elemental compositions for ash, wt % | | | | | |
|---|---|---|---|---|---|---|---|
| | wt % | Si | Zn | S | Ca | Al | Fe |
| Tyre char | 13.3 | 12.0 | 30.0 | 10.3 | 4.7 | 1.2 | 1.0 |

### 2. Preparation of Zinc Sulfate Catalysts

Pure ZnS, ZnO and ZnSO₄ were purchased from Merck Co Ltd. A certain amount of Pd(NO₃)₂·2H₂O (Merck) was mixed with ZnSO₄·7H₂O (Merck) and 30 mL ethanol in a liquid to solid (L/S) ratio of 10 mL/g in a beaker for ultrasonic treatment for 15 min. Then the mixture was transferred to an autoclave for hydrothermal treatment for 12 h at 180° C. and then vacuum dried at 80° C. After that, the powder was calcinated at 550° C. for 2 h for future use. The prepared catalyst was labelled as ZnSO₄/PdO whose PdO content was around 0.8 wt % (1.1 mol %).

### 3. Production of Furfural

### 3.1 Materials

D-allose (C₆H₁₂O₆), D-glucose (C₆H₁₂O₆) and xylan ((C₅H₁₀O₅)ₙ) tested in this study as feedstocks were reagent grade with a purity >99% and were purchased from Merck Co Ltd.

### 3.2 Pyrolysis Experiments

Pyrolysis experiments were carried out in a Pyro-GC system with a fixed-bed quartz tube reactor (2 mm in diameter and 30 mm in length), as has been previously described [13]. In brief, a biomass component and catalysts (with and without H₂O) were located at the first stage and second stage, respectively. The temperature varied in the range of 300-500° C. and the carrier gas is helium (He, 26 mL/min). The holding time is set at 25 s for each terminal temperature. The effluents discharged from the reactor were sent to an FID detector (Agilent, 7890 B) coupled with a capillary column for liquid products. The oven was ramped from 50° C. (on hold for 2 min) to 250° C. (on hold for 2 min) at 25° C./min. The split ratio is set at 25:1. At least two repeats were carried out for each condition.

### 3.3 Results

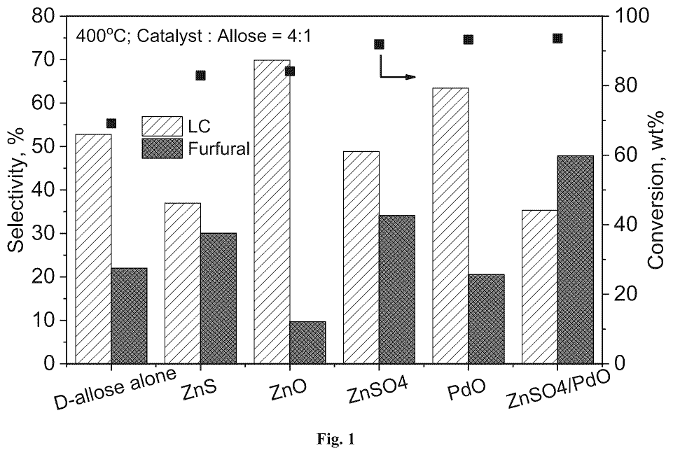

3.3.1 D-Allose as Feedstock Different catalysts were tested for the catalytic pyrolysis of D-allose (C6) at 400° C. with a catalyst to biomass mass ratio of four. The results are shown in FIG. 1. The conditions were as follows: Pyrolysis in a Pyro-GC system; Feedstock: D-allose; Pyrolysis temperature: 400° C.; Heating rate: 20° C./ms; Holding time: 25 s; Catalyst to D-allose mass ratio: 0 or 4; Water to D-allose mass ratio: 0.

In terms of the conversion of allose, it increased from 70 wt % for allose alone to 94 wt % when the ZnSO₄/PdO (0.8 wt % (1.1 mol %) PdO) catalyst was employed. In the order of blank case (i.e. allose alone), ZnS, ZnSO₄ and ZnSO₄/PdO, the selectivity of furfural increased from 22% for allose alone case to 30%, 34% and 48%, respectively. In contrast, the use of ZnO or PdO promoted the production of furan other than furfural.

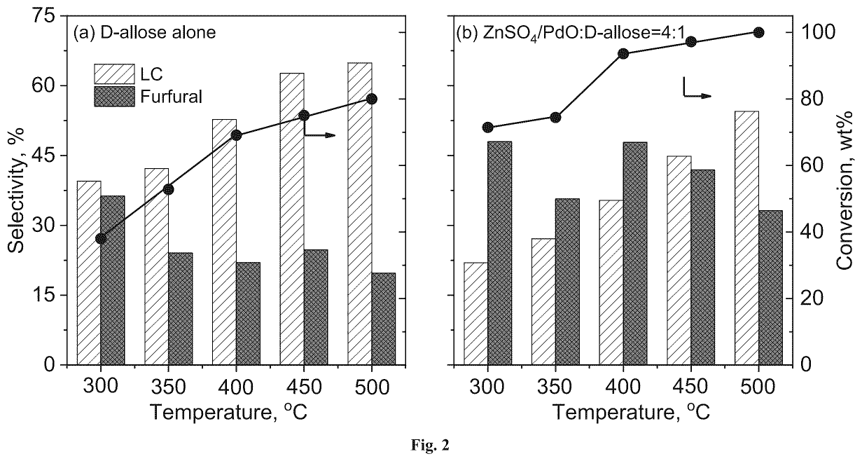

Considering that the temperature has a strong influence on the catalytic activity, different temperatures ranging from 300 to 500° C. were tested with and without the ZnSO₄/PdO as the catalyst. As seen in FIG. 2, compared with D-allose alone, the use of this catalyst increased both conversion of D-allose and selectivity of furfural for all the temperatures tested. In particular, 400° C. was further confirmed as the optimum temperature for both overall conversion of allose and the selectivity of furfural.

The conditions were as follows: Pyrolysis in a Pyro-GC system; Feedstock: D-allose; Pyrolysis temperature: 300-500° C. with a temperature difference of 50° C.; Heating rate: 20° C./ms; Holding time: 25 s; ZnSO₄/PdO to D-allose mass ratio: 0 or 4; Water to D-allose mass ratio: 0.

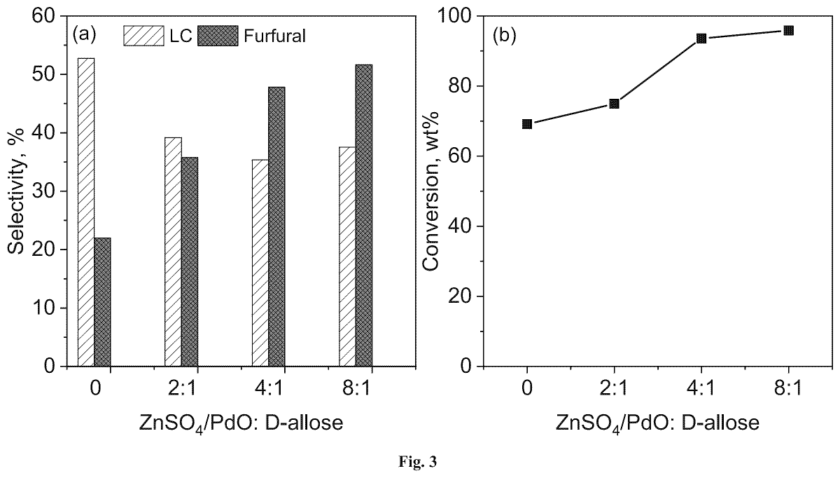

In addition, different catalyst/allose ratios were tested at 400° C. as shown in FIG. 3. The conditions were as follows: Pyrolysis in a Pyro-GC system; Feedstock: D-allose; Pyrolysis temperature: 400° C.; Heating rate: 20° C./ms; Holding time: 25 s; ZnSO₄/PdO to D-allose mass ratio: 0-8; Water to D-allose mass ratio: 0.

The selectivity of furfural increased linearly when 2 and 4 times ZnSO₄/PdO catalysts were used and then gradually increased to around 52% when eight times catalysts were used. Similarly, the conversion of allose increased from 69 wt % to 96 wt % at a catalyst to allose mass ratio of eight.

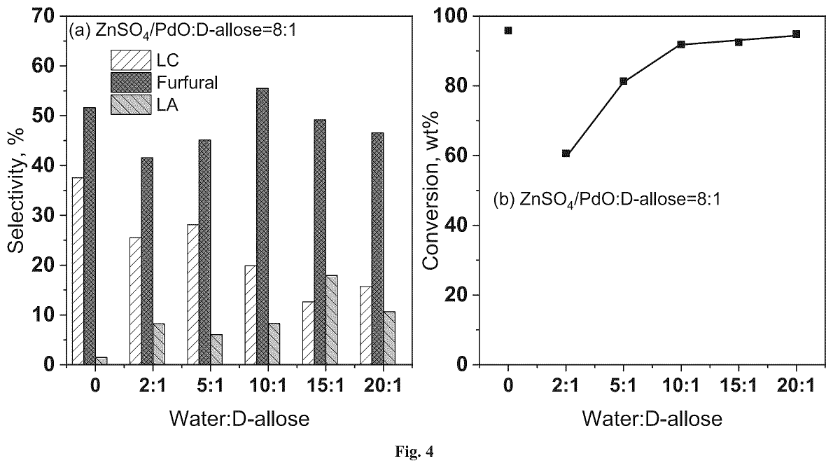

The effect of water vapour was studied by injecting different ratios of H₂O into the reactor at a ZnSO₄/PdO to allose mass ratio of eight. The results are shown in FIG. 4 The conditions were as follows: Pyrolysis in a Pyro-GC system; Feedstock: D-allose; Pyrolysis temperature: 400° C.; Heating rate: 20° C./ms; Holding time: 25 s; ZnSO₄/PdO to D-allose mass ratio: 8; Water to D-allose mass ratio: 0-20.

It was found that the steam promoted the formation of levulinic acid (LA). The production of light compound (LC) was prohibited by steam while the furfural selectivity showed an interesting trend of increase as the steam to allose ratio increased to 10:1. At the steam to allose ratio of 10, the selectivity for furfural was about 56% with a conversion of allose of 92 wt %.

### 3.3.2 D-Glucose as Feedstock

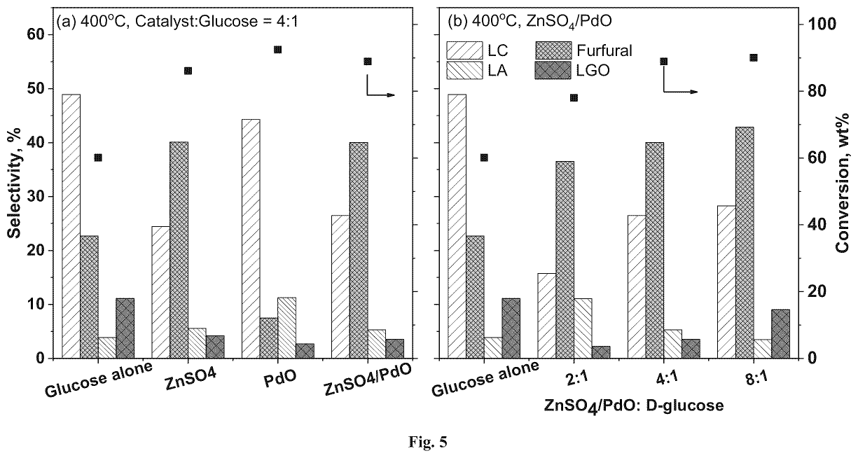

Zinc sulfate catalyst were also evaluated for their ability to convert glucose to furfural. Similar with the catalytic performance for D-allose, ZnSO₄/PdO was also found to be the best catalyst for the selectivity of furfural from glucose, as demonstrated in FIG. 5a. The conditions used were as follows: Pyrolysis in a Pyro-GC system; Feedstock: D-glucose; Pyrolysis temperature: 400° C.; Heating rate: 20° C./ms; Holding time: 25 s; Catalyst to D-glucose mass ratio: 0-8; Water to D-glucose mass ratio: 0.

By increasing the catalyst to glucose ratio, as shown in FIG. 5b, the furfural selectivity was increased from 23% for glucose alone case to 43% for ZnSO₄/PdO catalyst in the mass ratio of 8:1 to glucose. The furfural selectivity of glucose and allose was similar without catalyst, 23% and 22% respectively.

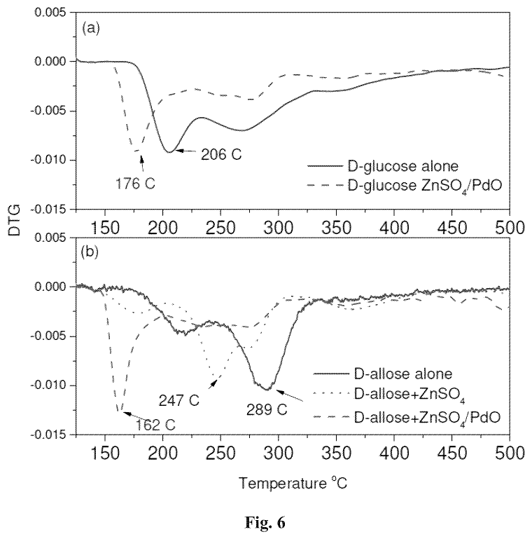

Thermogravimetric analysis (TGA, see FIG. 6) showed that the decomposition temperature decreased by 30° C. for glucose, compared to 127° C. for allose when ZnSO₄/PdO catalyst was used. This result supports that ZnSO₄/PdO is a particularly good catalyst for allose as feedstock.

### 3.3.4 Xylan as Feedstock

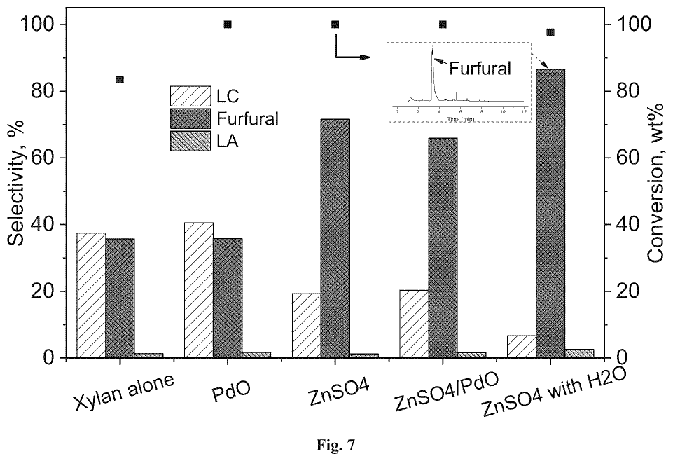

The use of zinc sulfate catalysts in catalysing the transformation of allose to furfural was also investigated. Different from the cases of using D-glucose (C6) and D-allose (C6) as feedstock for which ZnSO₄/PdO has the highest catalytic activity for the production of furfural, pure ZnSO₄ was found to be the best catalyst when xylan (C5) was used as the feedstock (see FIG. 7). The conditions used were as follows: Pyrolysis in a Pyro-GC system; Feedstock: Xylan; Pyrolysis temperature: 400° C.; Heating rate: 20° C./ms; Holding time: 25 s; Catalyst to xylan mass ratio: 4; Water to xylan mass ratio: 0 or 10.

The conversion of xylan was found to be close to 100% when using ZnSO₄ or ZnSO₄/PdO catalyst. Additionally, upon the use of ZnSO₄ catalyst, the selectivity of furfural increased from 36% for xylan alone to 72% with ZnSO₄ catalyst at a catalyst:xylan mass ratio of four to one. The furfural selectivity was further improved to 87% when the steam was added at a mass ratio of 10:1 catalyst to xylan.

### 3.3.5 Real Biomass as Feedstock—Sugarcane Bagasse and Corncob

ZnSO₄ and Pd-laden ZnSO₄ catalysts were also tested using real biomass including sugarcane bagasse (see Table 2) and corncob (see Table 3).

For sugarcane bagasse, ZnSO₄/PdO(0.4) yielded the highest amount of furfural, at 22.8 wt % with a selectivity 44 area % at 400° C. and a water to sugarcane bagasse mass ratio of 10. This furfural yield is about 2 times higher than the yield reported using Westpro/Huaxia Technology of 8-11 wt % [14]. Meanwhile, 14.6 wt % acetic acid can be produced.

For corncob, 32.2-32.9 wt % furfural was obtained with the catalyst containing 0.4-1.1 mol % PdO (ZnSO₄/PdO) and a water to corncob mass ratio of 10. This furfural yield is about 3 times higher than 10-12 wt % by Westpro/Huaxia Technology [14]. The overall liquid yield was in the range of 65.0-69.1 wt % and the selectivity of furfural varied from 58.8-61.2 area %. The main by-product was acetic acid, at 12.5-13.7 wt %.

The conditions used were as follows: Pyrolysis in a Pyro-GC system; Feedstock: sugarcane bagasse or corncob; Pyrolysis temperature: 400° C.; Heating rate: 20° C./ms; Holding time: 25 s; Catalyst to biomass mass ratio: 8; Water to biomass mass ratio: 0 or 10.

TABLE 2
Yield of different products obtained from pyrolysis of sugarcane bagasse at 400°
C. with different catalysts at a catalyst to sugarcane bagasse mass ratio of 8:1.

| | Y_{solid}, | Y_{liquid}, | S_{Furfural}, | Y_{Furfural}, | S_{Acetic acid}, | Y_{Acetic acid}, |
|---|---|---|---|---|---|---|
| | wt % | wt % | area % | wt. % | area % | wt % |
| No catalyst | 24.1 | 60.5 | 10.6 | 5.2 | 16.0 | 9.3 |
| ZnSO₄ | 21.2 | 58.93 | 27.2 | 13.0 | 18.6 | 11.3 |
| ZnSO₄ 7H₂O | 3.3 | 70.64 | 28.6 | 16.4 | 37.4 | 25.6 |
| ZnSO₄Pd(1.1) | 8.5 | 70.67 | 30.8 | 17.6 | 21.4 | 15.6 |
| ZnSO₄Pd(1.1) + | 15.6 | 69.61 | 32.5 | 18.3 | 38.9 | 26.4 |
| H₂O | | | | | | |
| ZnSO₄PdO(0.4) | 4.2 | 75.44 | 34.8 | 21.3 | 18.9 | 15.2 |
| ZnSO₄PdO(0.4) + | 6.6 | 64.08 | 44 | 22.8 | 20.0 | 14.1 |
| H₂O | | | | | | |

TABLE 3
Yield of different products obtained from pyrolysis of corncob at 400°
C. with different catalysts at a catalyst to corncob mass ratio of 8:1.

| | Y_{solid}, | Y_{liquid}, | S_{Furfural}, | Y_{Furfural}, | S_{Acetic acid}, | Y_{Acetic acid}, |
|---|---|---|---|---|---|---|
| | wt % | wt % | area % | wt. % | area % | wt % |
| No catalyst | 22.6 | 56.84 | 6.0 | 2.8 | 25.3 | 13.2 |
| ZnSO₄ | 7.4 | 69 | 15.9 | 8.9 | 22.6 | 15.0 |
| ZnSO₄ 7H₂O | 21.1 | 56 | 32.4 | 14.7 | 31.1 | 17.3 |
| ZnSO₄PdO(1.1) | 3.4 | 73.7 | 16.1 | 9.6 | 26.6 | 18.7 |
| ZnSO₄PdO(1.1) + | 12.2 | 69.1 | 58.8 | 32.9 | 16.1 | 13.7 |
| H₂O | | | | | | |
| ZnSO₄PdO(0.4) | 5 | 71.2 | 29.0 | 16.7 | 18.8 | 13.9 |
| ZnSO₄PdO(0.4) + | 10.7 | 65.0 | 61.2 | 32.2 | 15.3 | 12.5 |
| H₂O | | | | | | |

### 4. Conclusions

This study reported a novel process for producing zinc sulfate from waste materials, via a convenient process involving treating scrap tyre char with sulfuric acid, and isolating solid zinc sulfate from the leachate.

This study also reported a series of novel ZnSO₄ catalysts with and without loading PdO, processes for their production, and the use of such catalysts in processes for producing furfural from different biomass components by flash pyrolysis at 300-500° C. For C6 sugars, ZnSO₄/PdO was found to be the best catalyst compared with other catalysts in this study. The selectivity for furfural is about 56% with a D-allose conversion of 83.3 wt % at 400° C. with ZnSO₄/PdO as catalyst with steam (steam to allose=10:1 in mass).

When D-glucose was used as a feedstock, the furfural selectivity was 43% with 28.3% light compound (LC) at a D-glucose conversion of 90% with ZnSO₄/PdO as a catalyst.

In terms of the C5 sugar xylan, ZnSO₄ was found to be the best catalyst with the selectivity of furfural increasing from 36% for the case of xylan alone, to 72% with ZnSO₄ catalyst, and further up to 87% with the use of steam (steam to xylan=10:1 in mass). The conversion of xylan was close to 100% when ZnSO₄ or ZnSO₄/PdO was used as a catalyst.

For real biomass feedstock of sugarcane bagasse and corncob, 22.8 wt % and 32.9 wt % furfural was obtained respectively with ZnSO₄/PdO catalyst at a steam to biomass mass ratio of 10.

### REFERENCES

[1] Zhang L, Xi G, Chen Z, Jiang D, Yu H, Wang X. Highly selective conversion of glucose into furfural over modified zeolites. Chemical Engineering Journal 2017; 307:868-76.
[2] Gurbuz E I, Wettstein S G, Dumesic J A. Conversion of hemicellulose to furfural and levulinic acid using biphasic reactors with alkylphenol solvents. ChemSusChem 2012; 5(2):383-7.
[3] Agirrezabal-Telleria I, Gandarias I, Arias P L. Heterogeneous acid-catalysts for the production of furan-derived compounds (furfural and hydroxymethylfurfural) from renewable carbohydrates: A review. Catalysis Today 2014; 234:42-58.
[4] Alonso D M, Wettstein S G, Mellmer M A, Gurbuz E I, Dumesic J A. Integrated conversion of hemicellulose and cellulose from lignocellulosic biomass. Energy & Environmental Science 2013; 6(1):76-80.
[5] Mehdi Dashtban A G, Pedram Fatehi. Production of furfural: overview and challenges. Journal of Science & Technology for Forest Products and Processes 2012; 2:44-53.
[6] Gravitis J, Vedernikov N, Zandersons J, Kokorevics A, Mochidzuki K, Sakoda A, et al. Chemicals and biofuels from hardwoods, fuel crops and agricultural wastes. 2000:https://www.esf.edu/outreach/pd/2000/cellulose/gravitis.pdf.
[7] Heide E V D, Zhang T. Method for producing furfural from lignocellulosic biomass material. US 2011/0144359 A1.
[8] Sabesan S S, Christina Jacy. Production of furfural from biomass. US 2013/0109869 A1.
[9] Burket C, Hutchenson K W. Furfural production from biomass. US 2014/0171664 A1.
[10] Luo Y, Li Z, Li X, Liu X, Fan J, Clark J H, et al. The production of furfural directly from hemicellulose in lignocellulosic biomass: A review. Catalysis Today 2019; 319:14-24.
[11] Earthbound Report. What can the world do with 1.5 billion waste tyres, 2017.
[12] Paul T. Williams. Pyrolysis of Waste tyres: A review. Waste Management, 2013, Vol. 33 (8), 1714-1728.
[13] Zhou Q, Yang S, Wang H, Liu Z, Zhang L. Selective deoxygenation of biomass volatiles into light oxygenates catalysed by S-doped, nanosized zinc-rich scrap tyre char with in-situ formed multiple acidic sites. Applied Catalysis B: Environmental 2021; 282.
[14] Win, D. T., Furfural—gold from garbage. AU J. T., 2005. 8(4): p. 185-190.

The entire contents of each of the above-referenced documents is incorporated herein by reference in their entirety.

1. A process for producing furfural comprising the steps of:
pyrolyzing a lignocellulosic material or a fraction thereof and producing furfural;
wherein the production of furfural is catalysed by a solid zinc sulfate-rich catalyst.

2. The process of claim 1, wherein the catalyst comprises at least 50 wt % zinc sulfate.

3. The process of claim 1 or 2, wherein the solid zinc sulfate-rich catalyst contains a further transition metal.

4. The process of claim 3, wherein the further transition metal is palladium, iron, copper, cobalt or nickel.

5. The process of claim 3 or 4, wherein the further transition metal is palladium.

6. The process of any of claims 3-5, wherein the solid zinc-sulfate rich catalyst contains palladium metal and/or palladium oxide.

7. The process of any of claims 4 to 6, wherein the solid zinc sulfate-rich catalyst contains from 0.2 to 5 wt % further transition metal.

8. The process of claim 1 or 2, wherein the catalyst consists essentially of zinc sulfate.

9. The process of any of claims 1-8, wherein the lignocellulosic material or fraction thereof is selected from the group consisting of wood chips, sawdust, sugar cane, corncobs, bagasse, oat hulls, cottonseed hulls, rice hulls and wheat bran.

10. The process of any of claims 1-8, wherein the lignocellulosic material or fraction thereof is selected from the group consisting of a cellulosic fraction and a hemicellulosic fraction.

11. The process of any of claims 1-8, wherein the lignocellulosic material or fraction thereof is selected from the group consisting of a monosaccharide, a disaccharide and an oligosaccharide.

12. The process of claim 11, wherein the lignocellulosic material or fraction thereof is selected from the group consisting of allose, glucose and xylan.

13. The process of any of claims 1-12, wherein prior to pyrolysis, the lignocellulosic material or fraction thereof is subjected to one or more pre-processing steps to:
remove a lignin fraction and/or
hydrolyse saccharide linkages.

14. The process of any of claims 1-13, wherein the production of furfural is carried out in the absence of steam.

15. The process of any one of claims 1-13, wherein the production of furfural is carried out in the presence of steam.

16. The process of claim 15, wherein the weight ratio of steam to the lignocellulosic material or fraction thereof is in the range of from about 0.1:1 to about 20:1, optionally from about 8:1 to about 20:1.

17. The process of any of claims 1-16, wherein pyrolysis and/or production of furfural is carried out at a temperature in the range of from about 300 to about 500° C.

18. The process of any one of claims 1-17, wherein the process is carried out as a batch process.

19. The process of any one of claims 1-17, wherein the process is carried out as a continuous or semi-continuous process.

20. The process of any of claims 1-19, wherein the process comprises separating furfural from other reaction products.

21. The process of claim 20, wherein furfural is separated by distillation.

22. The process of claim 20, wherein furfural is separated by selective condensation of a product vapour stream containing furfural.

23. Furfural produced by a process according to any of claims 1-22.

24. A process for producing one or more of furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin, comprising:
producing furfural in accordance with any of claims 1 to 23; and
converting the furfural into furfuryl alcohol, furoic acid, furan, tetrahydrofuran, levulinic acid, butadiene, hexamethylenediamine, tetrahydrofurfuryl alcohol, methyltetrahydrofuran and furfural-phenolic resin.

25. A process for producing solid zinc sulfate comprising the steps of:
contacting tyre char with aqueous sulfuric acid and producing a mixture of aqueous zinc sulfate and solid tyre char residue;
separating aqueous zinc sulfate from solid tyre char residue; and
recovering solid zinc sulfate from the aqueous zinc sulfate.

26. The process as claimed in claim 25, wherein the step of contacting tyre char with aqueous sulfuric acid is carried out at a temperature in the range of from about 25 to about 90° C.

27. The process as claimed in claim 25 or 26, wherein the aqueous sulfuric acid has a sulfuric acid concentration in the range of from about 1 to about 5 moles per litre and used at a mass ratio in the range of from 1.5:1 to 10:1 aqueous sulfuric acid to tyre char.

28. The process as claimed in any of claims 25-27, wherein aqueous zinc sulfate is separated from solid tyre char residue by filtration.

29. The process as claimed in any of claims 25-28, wherein solid zinc sulfate is recovered by one or more of evaporation, crystallisation and precipitation.

30. The process as claimed in claim 29, wherein solid zinc sulfate is recovered by evaporation of water from aqueous sulfate to reduce the water content, at a temperature in the range of from 50 to 100° C., and subsequent precipitation of solid zinc sulfate.

31. The process as claimed in claim 30, wherein the reduction of water content during evaporation is controlled so that subsequent precipitation produces solid zinc sulfate heptahydrate.

32. The process as claimed in claim 29, wherein solid zinc sulfate is recovered by crystallization from water or an aqueous solvent, and the amount of water used is controlled so that crystallization produces solid zinc sulfate heptahydrate.

33. The process as claimed in any of claims 25-32, comprising the step of calcining solid zinc sulfate.

34. The process as claimed in claim 33, wherein the calcining step is carried out at a temperature in the range of from about 400 to about 800° C.

35. A process for producing a transition metal-doped zinc sulfate, comprising:
contacting tyre char with aqueous sulfuric acid and producing a mixture of aqueous zinc sulfate and solid tyre char residue;
separating aqueous zinc sulfate from solid tyre char residue;
recovering solid zinc sulfate from the aqueous zinc sulfate;
contacting zinc sulfate with a salt of a further transition metal; and
producing a transition metal-doped solid zinc sulfate.

36. The process as claimed in claim 35, wherein the further transition metal is palladium, iron, cobalt or nickel.

37. The process as claimed in claim 35 or 36, wherein solid zinc sulfate is recovered from the aqueous zinc sulfate prior to contacting with a salt of a further transition metal salt.

38. The process as claimed in claim 37, wherein the solid zinc sulfate is mixed with a salt of a further transition metal in the presence of an organic solvent.

39. The process as claimed in claim 38, wherein the organic solvent is ethanol.

40. The process as claimed in any of claims 37-39, wherein solid zinc sulfate is mixed with the salt of a further transition metal using ultrasonication.

41. The process as claimed in any of claims 36-40, wherein solid zinc sulfate is mixed with the salt of a further transition metal present in a solvent-soluble form so as to impregnate the zinc sulfate with the further transition metal.

42. The process as claimed in any of claims 35-41, wherein organic solvent is removed by heating and/or vacuum drying.

43. The process as claimed in claim 37, wherein the salt of a further transition metal is vaporised and then condensed onto the surface of solid zinc sulfate.

44. The process as claimed in claim 35, wherein the salt of a further transition metal is mixed with aqueous zinc sulfate and transition metal-doped solid zinc sulfate is produced by coprecipitation and/or cocrystallization.

45. The process as claimed in any of claims 35 to 44, wherein a stabilising and/or acidity-adjusting metal salt is added.

46. The process as claimed in claim 45, wherein solid zinc sulfate is recovered from the aqueous zinc sulfate prior to mixing with the salt of the further transition metal and with the stabilising and/or acidity-adjusting metal salt.

47. The process as claimed in claim 45, wherein the stabilising and/or acidity-adjusting metal salt is mixed with aqueous zinc sulfate and with the salt of the further transition metal, and transition metal-doped solid zinc sulfate is produced by coprecipitation and/or cocrystallization.

48. The process as claimed in any of claims 35 to 47, comprising the step of calcining the transition metal-doped solid zinc sulfate.

49. The process as claimed in claim 48, wherein the calcining step is carried out at a temperature in the range of from about 400 to about 800° C.

50. The process as claimed in any of claims 35-49, wherein, following calcination, the transition metal-doped solid zinc sulfate is subjected to a reduction step, by exposure to hydrogen at a temperature in the range of from about 400 to about 800° C.

51. Use of a zinc sulfate produced according to a process as claimed in any of claims 25-34, or of a transition metal-doped zinc sulfate produced according to a process as claimed in any of claims 35-50, as a catalyst for producing furfural from lignocellulosic material or a fraction thereof.

52. A process for regenerating an active transition metal-doped zinc sulfate catalyst which has been used for producing furfural from lignocellulosic material or a fraction thereof, comprising subjecting the transition metal-doped zinc sulfate to a reduction step, by exposure to hydrogen at a temperature in the range of from about 400 to about 800° C.

53. Solid zinc sulfate produced by a process according to any of claims 25-34.

54. A transition metal-doped solid zinc sulfate, wherein the transition metal is selected from the group consisting of palladium, iron, nickel and cobalt.

55. The transition metal-doped solid zinc sulfate as claimed in claim 54, which contains at least 50 wt % zinc sulfate.

56. The transition metal-doped solid zinc sulfate as claimed in claim 54 or 55, wherein the transition metal is palladium.

57. The transition metal-doped solid zinc sulfate as claimed in claim 56, which contains palladium metal and palladium oxide.

58. The transition metal-doped solid zinc sulfate as claimed in claim 56 or 57, which contains from 1 to 5 wt % transition metal.

59. The transition metal-doped solid zinc sulfate as claimed in any of claims 54-58, which contains a stabilising and/or acidity-adjusting additional metal.

60. Transition metal-doped solid zinc sulfate produced according to any of claims 35 to 50.
